SEQUENCE LISTING (28 sequences)

SEQ ID NO: 1 - protein, 25 aa, Artificial
  Signal peptide
MGVLLTQRTLLSLVLALLFPSMASM

SEQ ID NO: 2 - protein, 125 aa, Human
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 3 - protein, 25 aa, Artificial
  Linker
GGGGSGGGGSGGGGSGGGGSGGGGS

SEQ ID NO: 4 - protein, 452 aa, Artificial
  anti-IL-17A mAb hc-A
QVQLKESGPGLVQPSQTLSLTCTVSGFSLTNYYVHWVRQPPGKGLEWMGGVWNDGDTSYNSVLRSRLSITRDTSKSQVLLKMSSLQTEDTATYYCAREGREGFVGYYVMDAWGPGASVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 5 - protein, 214 aa, Artificial
  anti-IL-17A mAb lc-A
DIQMTQSPSFLSASVGERVTLSCKASQNINKYLDWFQQKLGEAPKLLIYNADNLHTGIPSRFSGSGSFSDFILTISSLQPEDDATYFCLQRESWPYTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - protein, 602 aa, Artificial
  Human CTLA4 ECD -linker- anti-IL-17A mAb hc-A
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDGGGGSGGGGSGGGGSGGGGSGGGGSQVQLKESGPGLVQPSQTLSLTCTVSGFSLTNYYVHWVRQPPGKGLEWMGGVWNDGDTSYNSVLRSRLSITRDTSKSQVLLKMSSLQTEDTATYYCAREGREGFVGYYVMDAWGPGASVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 7 - DNA, 1806 bp, Artificial
  Human CTLA4 ECD - linker - anti-IL-17A mAb hc-A encoding nucleotide sequence
gctatgcacgtcgctcagccagcagtggtcctggcatccagcagaggcatcgccagtttcgtgtgcgagtacgcttcacccggaaaggcaacagaagtcagagtgactgtcctgcgacaggcagacagccaggtgaccgaggtctgcgccgctacatatatgatgggcaacgaactgacatttctggacgattctatctgtactggcacctctagtggaaaccaagtgaatctgactatccagggcctgcgcgctatggataccggactgtacatttgcaaggtggagctgatgtatccccctccatactatctggggattggtaatggcacccaaatatacgtgattgacccagagccctgtcctgactctgatggaggtggaggatccggaggtggaggaagcgggggtggcggatctggaggaggagggtctggcggcggaggaagtcaggtgcaactgaaggagagcggccccggactggtgcaaccttcccaaacactcagcctcacctgtaccgtctccggattctccctgaccaactattacgtccactgggtcagacagcctcccggcaagggactggagtggatgggaggcgtgtggaatgacggcgacacctcctacaacagcgtcctcaggagcagactgagcattaccagagacaccagcaagagccaggtcctcctcaagatgtcctccctccagacagaagacaccgccacctattactgcgccagggaaggcagggaaggcttcgtcggctactatgtgatggatgcttggggccctggagctagcgtgacagtgagctccgcaaagaccacagcaccaagcgtgtacccactggcacctgtctgcggcgacactaccggatccagtgtgacactgggatgtctggtcaagggctatttccccgagcctgtgactctgacctggaatagcgggtccctgtcaagcggagtgcacacatttcccgctgtcctgcagtccgacctgtatactctgtcctctagtgtgacagtcacttcaagcacctggccctctcagagtattacatgcaacgtggcccatcctgcttcctctactaaggtggataagaaaatcgaaccaagaggccccaccattaagccttgtccaccctgcaaatgtccagcaccaaatctgctgggaggaccaagcgtgttcatctttcctccaaagatcaaggacgtgctgatgatctcactgagcccaattgtgacctgcgtggtcgtggacgtgagcgaggacgatcccgatgtgcagatcagctggttcgtcaacaatgtggaagtccacaccgcccagacccagacacatagagaggattacaactctacactgagggtcgtgagtgctctgcccattcagcatcaggactggatgagcgggaaggagttcaagtgcaaagtgaacaacaaggacctgcctgctccaatcgagaggaccattagcaagcctaaaggcagcgtgcgggcaccacaggtgtatgtcctgccccctccagaggaagagatgaccaagaaacaagtgactctgacctgtatggtcacagacttcatgcccgaagatatctacgtggagtggacaaacaacggaaagactgaactgaactacaagaacaccgagcctgtgctggactccgatggctcttactttatgtatagcaagctgcgcgtggaaaagaaaaactgggtcgagcgaaattcctactcttgtagtgtggtccacgaagggctgcataatcatcatactaccaagtctttttccagaaccccaggcaaa

SEQ ID NO: 8 - DNA, 642 bp, Artificial
  anti-IL-17A mAb lc-A encoding nucleotide sequence
gacatccagatgacacagtcccccagcttcctgagcgctagcgtgggagagagggtgaccctgagctgcaaggccagccagaacatcaacaagtacctggactggttccagcagaagctgggcgaagcccccaagctgctgatctacaacgccgacaacctccataccggcattcccagcaggttcagcggctccggctccttcagcgacttcatcctgaccatcagcagcctgcagcccgaggatgacgccacctacttctgcctgcagagggaatcctggccttacaccttcggcgccggcaccaagctggaactgaagagagctgacgctgcccctaccgtctccatctttcccccctccagcgagcaactcacaagcggcggcgcttccgtcgtgtgcttcctgaacaacttctaccccaaggacatcaacgtgaagtggaagatcgacggcagcgaaaggcagaacggcgtcctgaacagctggaccgaccaggactccaaggactccacctactccatgtcctccaccctcacactgaccaaggatgagtacgagaggcacaacagctacacctgcgaggccacacacaaaacatccaccagccccatcgtgaagtccttcaaccggaatgagtgc

SEQ ID NO: 9 - DNA, 27 bp, Artificial
  Primer
cccaagcttgccaccatgggggtcctg

SEQ ID NO: 10 - DNA, 27 bp, Artificial
  Primer
ccggaattctcatttgcctggggttct

SEQ ID NO: 11 - DNA, 24 bp, Artificial
  Primer
ccggaattctcagcactcattccg

SEQ ID NO: 12 - protein, 454 aa, Artificial
  anti-IL-17A mAb hc-B
QVQLQESGPGLVKPSETLSLTCTVSGFSLPSHSVSWIRQPPGKGLEWIGIIWNQGGTDYNSAFKSRVTISVDTSKNQFSLKLSSVTAADTAVYYCARNAYITDYYYENYFMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 220 aa, Artificial
  anti-IL-17A mAb lc-B
DIVMTQSPLSLPVTPGEPASISCKSSQSLLFSENQKNYLAWYLQKPGQSPQLLIYWTSTRQSGVPDRFSGSGSGTDFTLKISRVEAEDVGVYYCQQSYYTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 604 aa, Artificial
  Human CTLA4 ECD -linker- anti-IL-17A mAb hc-B
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDGGGGSGGGGSGGGGSGGGGSGGGGSQVQLQESGPGLVKPSETLSLTCTVSGFSLPSHSVSWIRQPPGKGLEWIGIIWNQGGTDYNSAFKSRVTISVDTSKNQFSLKLSSVTAADTAVYYCARNAYITDYYYENYFMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1812 bp, Artificial
  Human CTLA4 ECD -linker- anti-IL-17A mAb hc-B encoding nucleotide sequence
gctatgcacgtcgctcagccagcagtggtcctggcatccagcagaggcatcgccagtttcgtgtgcgagtacgcttcacccggaaaggcaacagaagtcagagtgactgtcctgcgacaggcagacagccaggtgaccgaggtctgcgccgctacatatatgatgggcaacgaactgacatttctggacgattctatctgtactggcacctctagtggaaaccaagtgaatctgactatccagggcctgcgcgctatggataccggactgtacatttgcaaggtggagctgatgtatccccctccatactatctggggattggtaatggcacccagatctacgtgattgacccagagccctgtcctgactctgatggaggtggaggatctggaggtggaggaagcgggggtggcggatccggaggtggaggaagcgggggtggcggatctcaggtccagctccaagagtccggccctggcctggtcaagcctagcgagaccctgtccctcacatgcaccgtgagcggctttagcctgccctcccattccgtgtcctggattaggcagccccccggcaaaggactggagtggatcggcatcatctggaaccagggcggaaccgactacaatagcgccttcaagagccgggtgaccatctccgtggacaccagcaagaaccagtttagcctgaagctgtccagcgtcacagccgccgacacagctgtctactactgcgcccggaacgcctacatcacagactactactacgagaactacttcatggacgcctggggacagggcacactggtgaccgtgtcctccgcctccaccaaaggacctagcgtgtttcccctcgcccccagcagcaaatccacctccggaggaaccgctgccctgggatgtctggtcaaggactacttccccgagcccgtgaccgtgtcctggaacagcggcgccctcacatccggcgtccacacatttcccgccgtgctgcaaagcagcggcctgtactccctgagcagcgtggtgaccgtgccttcctccagcctgggaacccagacctacatctgcaacgtgaaccacaagcctagcaacaccaaggtggacaagaaagtggagcccaagagctgcgacaagacccatacctgtcccccttgccctgcccctgaactgctgggaggccctagcgtgttcctgtttccacccaagcccaaagacacactgatgatttctcgcacacccgaagtcacttgtgtggtcgtggacgtgtcccacgaggatcctgaagtcaagttcaactggtacgtggatggcgtcgaggtgcataatgctaagaccaaacccagagaggaacagtacaacagcacctatcgcgtcgtgtctgtcctgacagtgctgcaccaggattggctgaacggaaaggagtacaagtgcaaagtgagcaacaaggccctgcccgctcctatcgagaagaccatttctaaggctaaaggccagcctagagaaccacaggtgtatacactgcctccaagtcgcgacgagctgacaaaaaaccaggtctccctgacttgtctggtgaagggattctaccctagcgatatcgcagtggagtgggaatctaatgggcagccagaaaacaattataagaccacaccccctgtgctggactcagatggttccttctttctgtacagtaaactgaccgtggacaagtccaggtggcagcaggggaacgtcttttcctgcagcgtgatgcatgaggccctgcacaatcattacacacagaaatctctgagtctgtcaccaggaaag

SEQ ID NO: 16 - DNA, 660 bp, Artificial
  anti-IL-17A mAb lc-B encoding nucleotide sequence
gacatagtcatgacccagagccccctgtccctgcctgtgacacctggagagcccgcctccatctcctgtaagagctcccaaagcctgctgttcagcgagaaccagaagaactacctggcctggtatctgcaaaagcctggccagtccccccagctcctcatctactggacaagcacccggcaaagcggcgtccctgacaggttctccggaagcggctccggaaccgacttcaccctgaaaataagccgggtggaggctgaggacgtgggagtgtactactgccagcagtcctactacaccccctacaccttcggccagggcaccaaagtcgagatcaagcggaccgtcgccgctccctccgtgttcatctttccccccagcgatgagcagctgaagtccggcaccgctagcgtcgtgtgcctcctgaacaacttctacccccgggaggccaaggtccagtggaaggtcgacaacgccctccagtccggcaacagccaagagagcgtgacagagcaggatagcaaggactccacctacagcctgagcagcaccctgaccctcagcaaggccgactacgagaagcacaaggtctacgcttgcgaagtgacccatcagggactcagctcccccgtcaccaagagcttcaaccggggcgagtgc

SEQ ID NO: 17 - DNA, 32 bp, Artificial
  Primer
ccggaattctcactttcctggtgacagactca

SEQ ID NO: 18 - DNA, 28 bp, Artificial
  Primer
ccggaattctcagcactcgccccggttg

SEQ ID NO: 19 - protein, 364 aa, Artificial
  CTLA4 ECD -linker- anti-IL-17A mAb lc
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDGGGGSGGGGSGGGGSGGGGSGGGGSDIQMTQSPSFLSASVGERVTLSCKASQNINKYLDWFQQKLGEAPKLLIYNADNLHTGIPSRFSGSGSFSDFILTISSLQPEDDATYFCLQRESWPYTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 602 aa, Artificial
  anti-IL-17A mAb hc-A-linker-CTLA4 ECD
QVQLKESGPGLVQPSQTLSLTCTVSGFSLTNYYVHWVRQPPGKGLEWMGGVWNDGDTSYNSVLRSRLSITRDTSKSQVLLKMSSLQTEDTATYYCAREGREGFVGYYVMDAWGPGASVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGSGGGGSGGGGSGGGGSGGGGSAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 21 - protein, 364 aa, Artificial
  anti-IL-17A mAb lc-A-linker-CTLA4 ECD
DIQMTQSPSFLSASVGERVTLSCKASQNINKYLDWFQQKLGEAPKLLIYNADNLHTGIPSRFSGSGSFSDFILTISSLQPEDDATYFCLQRESWPYTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 22 - protein, 330 aa, Artificial [sequence omitted]

SEQ ID NO: 23 - protein, 106 aa, Artificial
  Human IgG1 light chain constant region
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 602 aa, Artificial
  The chimera CTLA4 ECD-linker- anti-IL-17A mAb hc of the bifunctional fusion protein A
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDGGGGSGGGGSGGGGSGGGGSGGGGSQVQLKESGPGLVQPSQTLSLTCTVSGFSLTNYYVHWVRQPPGKGLEWMGGVWNDGDTSYNSVLRSRLSITRDTSKSQVLLKMSSLQTEDTATYYCAREGREGFVGYYVMDAWGPGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 214 aa, Artificial
  The chimera anti-IL-17A mAb lc of the bifunctional fusion protein A
DIQMTQSPSFLSASVGERVTLSCKASQNINKYLDWFQQKLGEAPKLLIYNADNLHTGIPSRFSGSGSFSDFILTISSLQPEDDATYFCLQRESWPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 602 aa, Artificial
  The humanized analogue CTLA4 ECD-linker- anti-IL-17A mAb hc of the bifunctional fusion protein A
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDGGGGSGGGGSGGGGSGGGGSGGGGSEVQLVESGGGLVQPGRSLRLSCAASGFSLTNYAMHWVRQAPGKGLEWVSAIWNDGDIDYADSVEGRFTISRDNAKNSLYLQMNSLRAEDTAVYYCAKEGREGFVGYYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 214 aa, Artificial
  The humanized analogue anti-IL-17A mAb lc of the bifunction fusion protein A
DIQMTQSPSSLSASVGDRVTITCKASQNINKYLDWYQQKPGKAPKLLIYNADNLHTGVPSRFSGSGSGTDFTLTISSLQPEDVATYYCLQRESWPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 452 aa, Artificial
  The humanized analogue anti-IL-17A mAb hc of the bifunctional fusion protein A
EVQLVESGGGLVQPGRSLRLSCAASGFSLTNYAMHWVRQAPGKGLEWVSAIWNDGDIDYADSVEGRFTISRDNAKNSLYLQMNSLRAEDTAVYYCAKEGREGFVGYYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2016/0340422 A1
(43) Pub. Date: Nov. 24, 2016

(54) BIFUNCTIONAL FUSION PROTEIN, PREPARATION METHOD THEREFOR, AND USE THEREOF

(71) Applicant: BEIJING HANMI PHARMACEUTICAL CO., LTD., Beijing (CN)

(72) Inventors: Guoqiang Chen, Beijing (CN); Jiawang Liu, Beijing (CN); Nanmeng Song, Beijing (CN); Yaping Yang, Beijing (CN); Mi Young Cha, Beijing (CN)

(73) Assignee: BEIJING HANMI PHARMACEUTICAL CO., LTD., Beijing (CN)

(21) Appl. No.: 15/114,006

(22) PCT Filed: Jan. 28, 2015
(86) PCT No.: PCT/CN2015/071718

§ 371 Date: Jul. 25, 2016

(30) Foreign Application Priority Data
Jan. 28, 2014 (CN) 201410042808.6

(51) Int. Cl. C07K 16/24; C07K 14/705

(52) U.S. Cl. CPC C07K 16/244; C07K 14/70521; C07K 2319/00; A61K 38/00

(57) ABSTRACT

The present invention provides a bifunctional fusion protein comprising the extracellular region of CTLA4 and an anti-IL-17 antibody, a gene encoding the protein, a vector comprising the gene, a host cell comprising the vector, and a pharmaceutical composition containing the protein.

### FIELD OF THE INVENTION

The present invention relates to a bifunctional fusion protein, a preparation method therefor and use thereof. In particular, the present invention relates to a bifunctional recombinant fusion protein which can block B7/CD28 and IL-17A/IL-17RA signal pathways simultaneously, a gene encoding the bifunctional fusion protein, a vector comprising the gene, a host cell comprising the vector, and a pharmaceutical composition containing the bifunctional fusion protein.

### BACKGROUND

It is regarded as an effective way for treating chronic inflammation to regulate and inhibit T-cell functions. Cytotoxic T lymphocyte-associated antigen 4 (CTLA4), also called CD152, is a leucocyte differentiation antigen. Ligands of both CTLA4 and CD28 are B7 molecules, and CTLA4 after binding to a B7 molecule induces anergy of T-cells and is involved in negative regulation of immune responses. Abatacept (the trade name Orencia™, Bristol-Myers Squibb Company) which was marketed in 2005 and is useful for treating rheumatoid arthritis, is a monofunctional fusion protein which is formed by fusing two extracellular domains (BCD) of a CTLA4 molecule to a Fc fragment of human IgGl and competitively blocks the binding of B7 to a CD28 molecule so as to inhibit proliferation and activation of T-cells (Genovese M C, Becker J C, SchiffM, et al. N. Engl. J. Med., 2005, 353: 1114-1123).

Interleukin 17A (IL-17 or IL-17A), a pro-inflammatory cytokine, is mainly produced from Thl7 cells and is a most representative member of IL-17 family (Miossec P, Kolls J K. Nat. Rev. Drug. Discov., 2012, 11:763-776). IL-17A after binding to its receptor (IL-17RA), may induce various cells (such as fibroblasts, epithelial cells and endothelial cells) to express inflammatory cytokines and chemokines, and plays an important role in immune defense of a body ( Lin Y, Ritchea S. Logar A. Immunity, 2009,31: 799-810). However, overexpressed IL-17A may cause inflammatory diseases. Synovial tissues of a patient with rheumatoid arthritis excessively express IL-17A, leading to the production of an important inflammatory factor-interleukin 6, which may be inhibited by an IL-17A neutralizing antibody (Chabaud M, Durand J M, Buchs N, et al. Arthritis Rheum. 1999, 42: 963-70). It has been demonstrated in many animal models with autoimmune diseases that pathological development of an inflammation may be effectively inhibited by neutralizing IL-17A with an antibody (Lubberts E. Koenders M I, Oppers-Walgreen B, et al. Arthritis Rheum., 2004, 50: 650-659). At present, no antibody medicament associated with IL-17A has been approved to be marketed, but three medicaments are in a clinical research stage, namely Secukinumab (IL-17A-targeting antibody), Ixekizumab (IL-17A-targeting antibody) and Brodalumab (IL-17RA-targeting antibody). However, as shown by the results of clinical research of said medicaments, as to some chronic inflammatory diseases such as rheumatoid arthritis, these medicaments did not show an expected therapeutic effect (Kellner H, Ther Adv Musculoskelet Dis., 2013, 5: 141-152). Therefore, it might increase clinical benefits for patients with some chronic inflammations such as rheumatoid arthritis by using a treatment regime of combined administration.

Combined administration requires sequential injections of two or more antibodies, or preparation of antibodies into a same dosage form. However, in an aspect, sequential injections of antibodies will reduce treatment compliance of a patient and increase pains. In another aspect, since different antibodies have different physicochemical properties, it is very difficult or almost impossible to prepare different antibodies into a same dosage form.

In view of this, it is still necessary to develop a new-type therapeutic medicament which simultaneously blocks B7/CD28 and IL-17A/IL-17RA signal pathways.

### SUMMARY OF THE INVENTION

The present invention provides a bifunctional fusion protein which simultaneously blocks B7/CD23 and IL-17A/IL-17RA signal pathways, a gene encoding the bifunctional fusion protein, a vector comprising the gene, a host cell comprising the vector, and a pharmaceutical composition containing the bifunctional fusion protein.

The first aspect of the present invention relates to a bifunctional fusion protein, comprising the extracellular region (i.e. extracellular domain) of a CTLA4 molecule and a functional fragment which, neutralizes the activity of IL-17. In one embodiment, IL-17 is IL-17A.

In one embodiment, the bifunctional fusion protein further comprises a linker, wherein the extracellular domain of a CTLA4 molecule is linked to N-terminal or C-terminal of the functional fragment which neutralizes the activity of IL-17 via a linker. In one embodiment, the linker is 1-25 amino acids in length, e.g. 20, 21, 22, 23, 24 or 25 amino acids in length, e.g. the linker is an amino acid sequence as set forth in SEQ ID NO.3.

In one embodiment, the functional fragment which neutralizes the activity of IL-17 is an anti-IL-17 antibody or a functional fragment thereof, a chimeric antibody, a humanized antibody, a full humanized antibody, a single-chain antibody or a bispecific antibody. In one embodiment, the extracellular domain of a CTLA4 molecule is linked to N-terminal of a heavy chain or a light chain of an anti-IL-17 antibody via a linker. In some embodiments, the anti-IL-17 antibody is an IgG, IgA, IgD, IgE or IgM antibody, or a hybrid thereof. In some embodiments, the anti-IL-17 antibody is an IgG antibody.

In one embodiment, the extracellular domain of the CTLA4 molecule comprises an amino acid sequence as set forth in SEQ ID NO.2 or a functional fragment thereof, or an amino acid sequence having the same function derived from the sequence as described above through substitution, deletion or addition of one or more amino acid residues, such as 2, 3, 4, 5, 10, 15, 20. 30 and 50 amino acid residues, or an amino acid sequence having at least 70% identity thereto and having the same function, such as at least about 75%. 80%, 85%, 90%, 91%, 92%, 93%, 94%, 95%, 96%, 97%, 98%, 99%, 99.1%, 99.2%, 99.3%. 99.4%, 99.5% or 99.6% identity.

In one embodiment, the heavy chain sequence of the anti-IL-17 antibody comprises an amino acid sequence as set forth in SEQ ID NO.28, or an amino acid sequence having the same function derived from the sequence as described above through substitution, deletion or addition of one or more amino acid residues, such as 2, 3, 4, 5, 10, 15, 20, 30 and 50 amino acid residues, or an amino acid sequence having at least 70% identity thereto and having the same function, such as at least about 75%, 80%, 85%, 90%, 91%, 92%, 93%, 94%, 95%, 96%, 97%, 98%. 99%, 99.1%, 99.2%, 99.3%, 99.4%, 99.5% or 99.6% identity. The light chain sequence of the anti-IL-17 antibody comprises an amino acid sequence as set forth in SEQ ID NO.27, or an amino acid sequence having the same function derived from the sequence as described above through substitution, deletion or addition of one or more amino acid residues, such as 2, 3, 4, 5, 10, 15, 20, 30 and 50 amino acid residues, or an amino acid sequence having at least 70% identity thereto and having the same function, such as at least about 75%, 80%, 85%, 90%, 91%, 92%, 93%, 94%, 95%, 96%, 97%. 98%, 99%, 99.1%, 99.2%, 99.3%, 99.4%, 99.5% or 99.6% identity.

In one embodiment, the extracellular domain of the CTLA4 molecule as set forth in SEQ ID NO. 2 is linked to N-terminal of the heavy chain sequence of an anti-IL-17 antibody as set forth in SEQ ID NO.4 or 12 or to N-terminal of the light chain sequence of an anti-IL-17 antibody as set forth in SEQ ID NO. 5 or 13 via a linker as set forth in SEQ ID NO.3.

In one embodiment, the extracellular domain of the CTLA4 molecule as set forth in SEQ ID NO.2 is linked to C-terminal of the heavy chain sequence of an anti-IL-17 antibody as set forth in SEQ ID NO.4 or 12 or to C-terminal of the light chain sequence of an anti-IL-17 antibody as set forth in SEQ ID NO.5 or 13 via a linker as set forth in a SEQ ID NO.3.

In one embodiment, the amino acid sequence of the bifunctional fusion protein is selected from the group consisting of the following combinations: SEQ ID NOs.5 and 6, SEQ ID NOs.13 and 14, SEQ ID NOs.4 and 19, SEQ ID NOs.24 and 25, SEQ ID NOs.26 and 27, or an amino acid sequence having the same function derived from the sequence as described above through substitution, deletion or addition of one or more amino acid residues, such as 2, 3, 4, 5, 10, 15, 20, 30 and 50 amino acid residues, or an amino acid sequence having at least 70% identity thereto and having the same function, such as at least about 75%, 80%, 85%, 90%, 91%, 92%, 93%, 94%, 95%, 96%, 97%, 98%, 99%, 99.1%, 99.2%, 99.3%, 99.4%, 99.5% or 99.6% identity.

The second aspect of the present invention relates to a gene encoding the bifunctional fusion protein as described above.

In one embodiment, the encoding gene comprises nucleotide sequences as set forth in SEQ ID NOs.7 and 8, or SEQ ID NOs.15 and 16.

In some embodiments, nucleic acid sequences encoding a light chain and a heavy chain in the vector are present in different expression cassettes.

The third aspect of the present invention relates to a recombinant vector, comprising the above encoding gene operably linked therein.

In some embodiments, the vector is an eukaryotic cell expression vector. In some embodiments, the vector is the vector X0GC which has been modified to harbor two expression cassettes.

The fourth aspect of the present invention relates to a host cell, comprising the recombinant vector as described above.

The fifth aspect of the present invention relates to a method for preparing the bifunctional fusion protein as described above, comprising the following steps:

(1) operably linking the encoding gene as described above into an eukaryotic expression vector, and transfecting the same to a host cell for expression, or transfecting the recombinant vector as described above to a host cell for expression; and

(2) purifying the bifunctional fusion protein.

In one embodiment, the eukaryotic expression vector is X0GC. In one embodiment, the host cell is a HBK293-T cell or a CHO cell.

The sixth aspect of the present invention relates to a method for producing the bifunctional fusion protein as described above, comprising culturing the host cell as described above under appropriate conditions allowing for expression, and isolating and purifying the expressed protein.

The seventh aspect of tire present invention relates to a pharmaceutical composition, comprising the bifunctional fusion protein as described above or a bifunctional fusion protein produced according to the method as described above.

In one embodiment, the bifunctional fusion protein as described above or the pharmaceutical composition as described above is useful for tire prevention, treatment or amelioration of rheumatoid arthritis, psoriasis, psoriatic arthritis, ankylosing spondylitis, Crohn's disease, systemic lupus erythematosus, lupus nephritis, multiple sclerosis, autoimmune encephalomyelitis, glomerulonephritis, idiopathic thrombocytopenic purpura, primary Sjogren's syndrome, gout or organ transplantation.

In another embodiment, the bifunctional fusion protein as described above or the pharmaceutical composition as described above is useful for the prevention, treatment or amelioration of rheumatoid arthritis, psoriasis, psoriatic arthritis, ankylosing spondylitis and Crohn's disease.

The eighth aspect of the present invention relates to use of the bifunctional fusion protein as described above, a bifunctional fusion protein produced according to the method as described above or the pharmaceutical composition as described above in the manufacture of a medicament for the prevention, treatment or amelioration of a disease.

In one embodiment, the disease is selected from a group consisting of rheumatoid arthritis, psoriasis, psoriatic arthritis, ankylosing spondylitis, Crohn3 s disease, systemic lupus erythematosus, lupus nephritis, multiple sclerosis, autoimmune encephalomyelitis, glomerulonephritis, idiopathic thrombocytopenic purpura, primary Sjogren's syndrome, gout or organ transplantation.

The ninth aspect of the present invention relates to a method for preventing, treating or ameliorating a disease, comprising a step of administering a therapeutically effective amount of the bifunctional fusion protein as described above, a bifunctional fusion protein produced according to the method as described above or the pharmaceutical composition as described above to a subject in need thereof.

In one embodiment, the disease is selected from a group consisting of rheumatoid arthritis, psoriasis, psoriatic arthritis, ankylosing spondylitis, Crohn's disease, systemic lupus erythematosus, lupus nephritis, multiple sclerosis, autoimmune encephalomyelitis, glomerulonephritis, idiopathic thrombocytopenic purpura, primary Sjogren's syndrome, gout or organ transplantation.

The tenth aspect of the present invention relates to a kit which comprises the bifunctional fusion protein as described above, the nucleic acid molecule as described above, the vector as described above or the host cell as described above.

The present invention has the following advantages:

The experimental results of the present invention show that the bifunctional fusion protein of the present invention has a high expression level, strongly inhibits the secretion of inflammatory cytokines in in vitro experiments, and can effectively inhibit the development of arthritis in animal models. As known to one skilled in the art, the combination medication or fusion medication of some medicaments with different mechanisms may cause different effects, such as antagonism, irrelevance, accumulation or synergism, etc. Which interaction is presented by the combination medication or fusion medication of two or more medicaments can only be determined through specific experimental studies with a lot of work paid. Although it is likely to use the CTLA4 molecule and the anti-IL-17 antibody in combination to treat rheumatoid arthritis in the prior art, since a human body is complex, in particular, as to a systemic and immune disease such as rheumatoid arthritis of which pathogenesis is extremely complex and has not been understood thoroughly yet, even if medicaments are used in combination or in a fusion form, a fully accumulated or synergetic effect of the therapeutic effect achieved by each of medicaments with different mechanisms can also rarely be achieved. An irrelevant or even antagonistic effect is presented in many cases. An increased therapeutic effect on rheumatoid arthritis achieved by the bifunctional fusion protein of the present invention provides a candidate medicament which may have a more excellent therapeutic effect for the treatment for patients with rheumatoid arthritis, in particular patients with moderate and severe rheumatoid arthritis. Furthermore, the bifunctional fusion protein of the present invention simultaneously acts on two different targets for treating rheumatoid arthritis, reduces probability of treatment failure or a poor therapeutic effect of a therapy targeting a single target, and has an important economic significance and social benefits. Compared with the use of a monofunctional protein (e.g. CTLA4-Fc, the present invention could reduce production cost, decrease the volume and frequency of clinical administration and improve compliance of a subject, and has a promising application prospect in the prevention and treatment of immune diseases.

### BRIEF DESCRIPTION OF THE DRAWINGS

FIGS. 1: A and B show purification of the bifunctional fusion proteins A and B in the embodiments of the present invention with Protein A affinity chromatography, respectively.

FIGS. 2: A and B show purification of the bifunctional fusion proteins A and B in the embodiments of the present invention with ion exchange chromatography, respectively.

FIGS. 3; A and B show the monomer purities of the bifunctional fusion proteins A. and B in the embodiments of the present invention, analyzed with size exclusion high-performance liquid chromatography, respectively.

FIG. 4 shows binding affinities of the bifunctional fusion proteins A and C to human IL-17A.

FIG. 5 shows binding affinities of the bifunctional fusion proteins A and C to mice IL-17A.

FIG. 6 shows that the bifunctional fusion protein A inhibits IL-17A-induced secretion of GRO α from a HT-29 cell.

FIG. 7 shows the immunosuppressive activity of the bifunctional fusion protein B measured with a mixed lymphocyte reaction.

FIG. 8 shows the effect of a humanized analogue of the bifunctional fusion protein A on the reduction of rat CIA arthritis scores.

### DETAILED DESCRIPTION OF THE INVENTION

One objective of the present invention is to provide a bifunctional fusion protein comprising the extracellular domain of a CTLA4 molecule and a functional fragment which neutralizes the activity of IL-17. The extracellular domain of a CTLA4 molecule and the functional fragment which neutralizes the activity of IL-17 are operably linked, maintaining their respective spatial structures and exerting their respective physiological activity. The extracellular domain of a CTLA4 molecule and the functional fragment which neutralizes the activity of IL-17 may be directly fused to each other without affecting respective functions thereof. Furthermore, the extracellular domain of a CTLA4 molecule is linked to N-terminal or C-terminal of the functional fragment which neutralizes the activity of IL-17. Alternatively, other spacer sequences may be added therebetween, e.g. a linker.

As known to one skilled in the art, although the terms “comprise”, “comprises”, “comprising” or “comprised” is used when defining the bifunctional fusion protein, it docs not mean that any other sequences irrelevant therewith in function may be arbitrarily added to the bifunctional fusion protein. When preparing a fusion protein having a complex composition such as a bifunctional fusion protein, in order to maintain the spatial structure, bioactivity and cell expression level of each component of the fusion protein, and in order to fuse each component together or in order to enhance the hydrolysis-resistant ability of the fusion protein, when preparing the fusion protein, one skilled in the art would add one or more additional amino acid residues among each component or to both terminals of the fusion proteins if required. Therefore, if the bifunctional fusion protein is defined in a closed expression way, these situations would not be truly covered.

The term “extracellular domain of a CTLA4 molecule” as used herein refers to an extracellular domain of cytotoxic T lymphocyte-associated antigen 4 (CTLA4). In a certain embodiment, an extracellular domain (ECD) sequence of a human CTLA4 protein is set forth in SEQ ID NO.2, an amino acid sequence from position 37 to position 161 of the human CTLA4 protein (Genbank accession number NM_005214.4).

In some embodiments, the term “functional fragment which neutralizes the activity of IL-17” as used herein refers to an immune globulin against IL-17, or a modifier, a functional equivalent, a functional fragment or a variant thereof, in some embodiments, the functional fragment which neutralizes the activity of IL-17 is an IgG antibody raised against IL-17. In some embodiments, the IgG is a chimeric, humanized or full human IgG. In some embodiments, the modifier may be a chemical modifier such as an acylated, alkylated or PEGylate product, as long as these modifiers retain the ability to target IL-17. In some embodiments, the functional equivalent refers to other polypeptide fragments capable of achieving the target binding ability of the immune globulin to IL-17. In some embodiments, the functional fragment refers to a protein fragment which retains the ability to target IL-17. such as a single-domain antibody, a single-chain antibody, a single chain variable fragment (scFv), a Fab fragment or F(ab′)2 fragment. In some embodiments, the variant refers to a polypeptide derived from a parent protein through one or more changes, i.e. substitutions, insertions and/or deletions at one or more (some) positions. in some embodiments, the linker is 5-30 amino acids in length. In some embodiments, the linker is 25 amino acids in length, in some embodiments, the sequence of the linker is GGGGSGGGGSGGGGSGGGGSGGGGS. The linker as used herein does not have special restrictions, as long as it can play a role in spacing two components of the fusion protein so as to make each component correctly form its respective spatial structure, and retain the bioactivity and the cell expression level thereof.

In some embodiments, the amino acid sequence of the bifunctional fusion protein is selected from tire group consisting of the following combinations: SEQ ID NOs.5 and 6, SEQ ID NOs.13 and 14. SEQ ID NOs.4 and 19, SEQ ID NOs.24 and 25, SEQ ID NOs.26 and 27, or an amino acid sequence having the same function obtained from the sequence as described above through substitution, deletion or addition of one or more amino acid residues, such as 2, 3, 4, 5, 10, 15, 20, 30 and 50 ammo acid residues, or an amino acid sequence having at least 70% identity thereto and having the same function, such as at least about 75%, 80%, 85%, 90%, 91%, 92%. 93%. 94%, 95%, 96%, 97%, 98%, 99%, 99.1%, 99.2%, 99.3%. 99.4%, 99.5% or 99.6% identity. A conservative amino acid of an amino acid is well known in the art.

The term “identity” as used herein has a meaning as generally known in the art. One skilled in the art also well knows the rules and standards for determining identities between different sequences. The sequences of the present invention defined with different identity degrees must simultaneously have the activity of the extracellular domain of a CTLA4 molecule or the activity to neutralize IL-17. One skilled in the art well knows the processes and means for screening a variant sequence with the above activities. One skilled in the art would readily obtain such a variant sequence under the teaching of the disclosure of tire present invention.

In one embodiment, the present invention relates to an encoding gene comprising a nucleotide sequence encoding the bifunctional fusion protein as described above. As known to one skilled in the art, although the terms “comprise”, “comprises”, “comprising” or “comprised” are used when defining the encoding gene, it does not mean that any other sequences irrelevant therewith in function may be arbitrarily added to both terminals of the encoding gene. One skilled in the art knows that in order to meet the recombination operation requirements, it is necessary to add appropriate restriction endonuclease cleavage sites to both terminals of the encoding gene, or additionally add start codon, stop codons and the like. Therefore, if the encoding gene is defined in a closed expression way, these situations would not be truly covered.

As known to one skilled in the art, in the case that the encoded amino acid is not changed, one or more codons in the encoding gene sequence may be equivalently substituted, e.g. one or some codons such as 1, 2, 3, 4, 5, 6, 8, 9, 10, 15, 20, 30, 40 and 50 codons. Codon usage table is well known in the art.

In one embodiment, the present invention relates to a recombinant vector, comprising the encoding gene as described above which is operably linked therein. The recombinant vector may be a recombinant expression vector, prokaryotic or eukaryotic, but preferably a eukaryotic expression vector, more preferably a recombinant expression vector for mammalian eukaryotic expression.

The term “operably linked” used herein refers to such a linking mode, wherein the encoding gene is placed at an appropriate position to render the encoding gene correctly and successfully duplicated, transcribed or expressed.

In one embodiment, the present invention relates to a host cell into which the recombinant vector as described as above is transformed or transfected. The host cell includes a mammalian cell, a bacterial cell, a yeast cell, an insect cell and a plant cell.

In a preferred embodiment, the host cell includes a CHO cell, a HEK293 cell, a NSO cell and a SP2/0 cell.

In one embodiment, the present invention relates to a method for producing the bifunctional fusion protein, comprising the steps of culturing the above host cell under appropriate conditions for expression of a polypeptide, and isolating and purifying the expressed polypeptide from the cell medium. Preferably, the purified polypeptide has a purity of greater than 50%, more preferably greater than 60%, 65%, 70%, 75%, 80%, 85%, 90%, 95%. 96%, 97%, 98%, 99%, 99.5%, 99.6%, 99,7%, 99.8% or 99.9%.

In one embodiment, the present invention relates to a pharmaceutical composition, comprising tire bifunctional fusion protein as described above and a pharmaceutically acceptable carrier. The bifunctional fusion protein may be the sole active ingredient, or may also be one of active ingredients of the pharmaceutical composition. Oilier active ingredients may be other therapeutic agents which can be used in combination with the bifunctional fusion protein.

In a preferred embodiment, the pharmaceutical composition of the present invention may be a single-dose dosage form, a topical dosage form or a systemic dosage form.

In one embodiment, the present invention relates to use of the bifunctional. fusion protein as described above or the pharmaceutical composition as described above in the prevention, treatment or amelioration of a disease selected from a group consisting of rheumatoid arthritis, psoriasis, psoriatic arthritis, ankylosing spondylitis. Crohn's disease, systemic lupus erythematosus, lupus nephritis, multiple sclerosis, autoimmune encephalomyelitis, glomerulonephritis, idiopathic thrombocytopenic purpura, primary Sjogren's syndrome, gout or organ transplantation. In some embodiments, the bifunctional fusion protein as described above or the pharmaceutical composition as described above is useful for the prevention, treatment or amelioration of a disease selected from a group consisting of rheumatoid arthritis, psoriasis, psoriatic arthritis, ankylosing spondylitis or Crohn's disease.

In some embodiments, the present invention relates to a method for preventing, treating or ameliorating a disease, comprising a step of administering a therapeutically effective amount of the bifunctional fusion protein as described above, the bifunctional fusion protein produced according to the method as described above or the pharmaceutical composition as described above to a subject in need thereof.

In one embodiment, the disease is selected from a group consisting of rheumatoid arthritis, psoriasis, psoriatic arthritis, ankylosing spondylitis, Crohn's disease, systemic lupus erythematosus, lupus nephritis, multiple sclerosis, autoimmune encephalomyelitis, glomerulonephritis, idiopathic thrombocytopenic purpura, primary sjogren's syndrome, gout or organ transplantation.

The term “therapeutically effective amount” refers to an amount that may exert a pharmacological effect in the body of a subject when administered thereto. “A therapeutically effective amount” could be readily determined by one skilled in the art according to the patient's conditions such as age, body weight and disease suite.

In one embodiment, the present invention relates to use of the bifunctional fusion protein as described above, the bifunctional fusion protein produced according to the method as described above or the pharmaceutical composition as described above in the manufacture of a medicament for the prevention, treatment or amelioration of a disease.

In one embodiment, the disease is selected from a group consisting of rheumatoid arthritis, psoriasis, psoriatic arthritis, ankylosing spondylitis. Crohn's disease, systemic lupus erythematosus, lupus nephritis, multiple sclerosis, autoimmune encephalomyelitis, glomerulonephritis, idiopathic thrombocytopenic purpura, primary Sjogren's syndrome, gout or organ transplantation.

As known to one skilled in the art, although the conditions which can be prevented, treated or ameliorated by the bifunctional fusion protein of the present invention have been listed above, the conditions which can be treated by the bifunctional fusion protein of the present invention are not limited to the above listed specific conditions. Any conditions which may he prevented, treated or ameliorated by simultaneously blocking B7/CD28 and IL-17A/IL-17RA signal pathways are falling within the scope of the present invention.

According to the above technical solutions of the present invention, the present invention has the following beneficial effects:

1) Blocking molecules of two signal pathways B7 and IL-17A (the extracellular domain of human CTLA4 and an anti-IL-17 antibody) are fused into a single molecule for expression and production. Compared with the production of extracellular domain of a CTLA4 protein and the anti-IL-17 antibody respective, it vastly reduces the operation and production cost.

2) When the extracellular domain of a human CTLA4 protein is linked to the anti-IL-17 antibody, the obtained protein of interest forms a tetramer. which not only meets the requirements for forming a dimer form to make the extracellular domain of CTLA4 exert its activity, but also keeps the titer of the anti-IL-17 antibody still divalent. Therefore, the total titer of the protein of interest is trivalence. Furthermore, as shown by the screening and comparison of the linking modes and in vitro binding assays, the operable linking mode in the present invention has no influence on the binding of the extracellular domain of the CTLA4 molecule to its ligand B7 and the binding of the anti-11,-17 antibody to IL-17.

3) After further modification, the bifunctional protein of the present invention may form a chimera or a humanized analogue, and further decrease its immunogenicity while maintaining the binding activity thereof.

4) The results of disease model experiments show that the bifunctional fusion protein can effectively relieve an inflammatory response of the disease animal model. The present invention is further illustrated hereby through the non-restrictive examples as below. As well known to one skilled in tire art, without deviation from the spirit of the present invention, one may make many modifications to tire present invention, and such modifications also fall into the scope of the present invention. if without a definite indication, the following experimental methods are conventional methods. If without a definite indication, the experimental materials used can be readily obtained from commercial companies.

### EXAMPLES

### Example 1: Construction of the Expression Vector of the Bifunctional Fusion Protein A

(1) The Amino Acid Sequences and Encoding Nucleotide Sequences of the Bifunctional Fusion Protein A

The signal peptide sequence used to express the bifunctional fusion protein A is set forth in SEQ ID NO.1; the extracellular domain sequence (ECD) of the human CTLA4 protein is set forth, in SEQ ID NO.2, which is an amino acid sequence from position 37 to position 161 of the human CTLA4 protein (Genbank accession number NM_005214.4); the linker is (G₄S)₅ which has a sequence as set forth in SEQ ID NO.3; the variable region sequence of the anti-IL-17A antibody (control antibody A) in the bifunctional fusion protein A is from U.S. Pat. No. 7,846,443 (rat 1D10), and the constant region sequence is a constant region sequence of mice IgG2a, i.e. the heavy chain (hc) sequence of the anti-IL-17A antibody is set forth in SEQ ID NO.4 and the light chain (hc) sequence is set forth in SEQ ID NO. 5; the amino acid sequence of the human CTLA4 ECD-linker-anti-IL-17AmAb hc-A is set forth in SEQ ID NO.6.

The nucleotide sequence which encodes the amino acid sequence as set forth in SEQ ID NO. 6 is optimized according to the codon bias of mammalian cells, and the sequence obtained is set forth in SEQ ID NO.7. The nucleotide sequence which encodes the amino acid sequence as set forth in SEQ ID NO.5 is optimized according to codon bias of mammalian cells, and the sequence obtained is set forth in SEQ ID NO.8. The sequences as set forth in SEQ ID NO.7 and SEQ ID NO.8 have been synthetized by GenScript (Nanjing) Co., Ltd., which is introduced into the pUC57 vector by utilizing TA-cloning. and the resulting positive clones have been sequenced, verified, and named as pUC57-CTLA4-anti-IL-17A mAb hc-A and pUC57-anti-IL-17AmAb Ic-A respectively.

(2) Construction of the Expression Vector of the Bifunctional Fusion Protein A and Preparation of Plasmids for Transfection

The pUC57-CTLA4-anti-IL-17A mAb hc-A plasmid (GenScript (Nanjing) Co., Ltd.) is used as a template. The sequence which encodes the signal peptide-human CTLA4 ECD-linker-anti-IL-17AmAb hc-A is amplified through the conventional PCR. The upstream primer used comprises a Hind IIII enzyme cutting site, and the sequence is CCCAAGCTTGCCACCATGGGGGTCCTG (SEQ ID NO.9). The downstream primer comprises a EcoR I enzyme cutting site, and the sequence is CCGGAATTCTCATTTGCCTGGGGTTCT (SEQ ID NO.10).

The pUC57-anti-IL-17A mAb Ic-A plasmid (GenScript (Nanjing) Co., Ltd.) is used as a template. The sequence which encodes anti-IL-17A mAb Ic-A is amplified through the conventional PCR. The upstream primer used comprises a Hind in enzyme cutting site, and the sequence is SEQ ID NO.9. The downstream primer comprises an EcoR I enzyme cutting site, and the sequence is CCGGAATTCTCAGCACTCATTCCG (SEQ ID NO.11).

The sequences encoding the signal peptide-human CTLA4 ECD-linker-anti-IL-17AmAb hc-A and the anti-IL-17A mAb Ic-A obtained by amplification (1881 bp and 717 bp in length, respectively) are isolated through 1% agarose gel electrophoresis and then the corresponding fragments are recovered. The gene fragments recovered are introduced info the eukaryotic expression vector X0GC (US publication NO. 20100120089) after being subjected to Hind III and EcoR I digestion, to obtain recombinant plasmids X0GC-CTLA4-anti-IL-17A mAb hc-A and X0GC-anti-IL-17A mAb Ic-A, which are then respectively transformed into Escherichia coli DH5 to obtain recombinant bacteria strains DH5 α/X0GC-CTLA4-anti-IL-17AmAb hc-A and DH5 α/X0GC-anti-IL-17AmAb Ic-A. Positive clones are screened through PCR and DNA sequencing is conducted so as to verify that the recombinant plasmids are correctly constructed.

The positive strains DH5 α/X0GC-CTLA4-anti-IL-17AmAb hc-A and DH5 α/X0GC-anti-IL-17 AmAb Ic-A are respectively seeded into IL LB/Amp liquid medium (composed of 1% peptone (BD company), 0.5% yeast extract (BD company) and 1% NaCl (Sinopharm Chemical Reagent Co., Lid.)), and incubated overnight with 180 rpm shaking speed at 37° C. On the second day, plasmids are extracted by using an EndoFree Plasmid Maxi Kit (Qiagen, 12381) for transfecting HEK293-T cells (Cell Resource Center of Shanghai Institutes for Biological Sciences, Chinese Academy of Sciences).

### Example 2: Construction of the Expression Vector of the Bifunctional Fusion Protein B

(1) The Amino Acid Sequences and Encoding Nucleotide Sequences of the Bifunctional Fusion Protein B

The signal peptide sequence used to express the bifunctional fusion protein B is set forth in SEQ ID NO.1; the ECD sequence of the human CTLA4 protein as set forth in SEQ ID NO.2 is an amino acid sequence from position 37 to position 161 of the human CTLA4 protein (Genbank accession number NM_005214.4); the linker is (G₄S)₅ which has a sequence as set forth in SEQ ID NO.3; the sequence of the anti-IL-17A antibody in the bifunctional fusion protein B is from U.S. Pat. No. 7,846,443 (humanized 16C10), of which the heavy chain (hc) sequence is set forth in SEQ ID NO.12 and the light chain (lc) sequence is set forth in SEQ ID NO.13; the amino acid sequence of the human CTLA4 ECD-linker-anti-IL-17AmAb hc-B is set forth in SEQ ID NO. 14.

The nucleotide sequence which encodes the amino acid sequence as set forth in SEQ. ID NO14 is optimized according to the codon bias of mammalian cells, and the sequence obtained is set forth in SEQ ID NO.15. The nucleotide sequence which encodes the amino acid sequence as set forth in SEQ ID NO.13 is optimized according to the codon bias of mammalian cells, and the sequence obtained is set forth in SEQ ID NO.16. The sequences as set forth in SEQ ID NO.15 and SEQ ID NO.16 have been synthetized by GenScript (Nanjing) Co., Ltd., which is introduced into the pUC57 vector by utilizing TA-cloning, and the resulting positive clones have been sequenced, verified and named as pUC57-CTLA4-anti-IL-17A mAb hc-B and pUC57-anti-IL-17AmAb Ic-B, respectively.

(2) Construction of the Expression Vector of the Bifunctional Fusion Protein B and Preparation of Plasmids for Transaction

The pUC57-CTLA4-anti-IL-17A mAb hc-B plasmid (GenScript (Nanjing) Co., Ltd.) is used as a template. The sequence which encodes the signal peptide-human CTLA4 ECD-linker-anti-IL-17AmAb hc-B is amplified through the conventional PCR, The upstream primer used comprises a Hind III enzyme cutting site, and the sequence is SEQ ID NO. 9. The downstream primer comprises a EcoR I enzyme cutting site, and the sequence is CCGGAATTCTCACTTTCCTGGTGACAGACTCA (SEQ ID NO. 17).

The pUC57-anti-IL-17A mAb Ic-B plasmid (GenScript (Nanjing) Co.. Ltd.) is used as a template. The sequence which encodes anti-IL-17A mAb lcB is amplified through the conventional PCR. The upstream primer used comprises a Hind III enzyme cutting site, and the sequence is SEQ ID NO. 9. The downstream primer comprises a EcoR I enzyme cutting site, and the sequence is CCGGAATTCTCAGCACTCGCCCCGGTTG (SEQ ID NO. 18).

The sequences encoding the signal peptide-human CTLA4 ECD-linker-anti-IL-17AmAb hc-B and the anti-IL-17A nAb Ic-B obtained by amplification (1887 bp and 735 bp in length, respectively) are isolated through 1% agarose gel electrophoresis and then the corresponding fragments are recovered. The gene fragments recovered are introduced into the eukaryotic expression vector X0GC (US publication No. 20100120089) after being subjected to Hind III and EcoR I digestion, to obtain recombinant plasmids X0GC-CTLA4-anti-IL-17A mAb hc-B and X0GC-anti-IL-17A mAb Ic-B, which are then respectively transformed into Escherichia coli DH5 α to obtain recombinant bacteria strains DM5 α/X0GC-CTLA4-anti-IL-17AmAb hc-B and DH5 a /X0GC-anti-IL-17AmAb Ic-B. Positive clones arc screened through PCR and DNA sequencing is conducted so as to verify that the recombinant plasmids are correctly constructed.

The positive strains DH5 α/X0GC-CTLA4-anti-IL 17AmAb hc-B and DH5 /X0GC-anti-IL-17AmAb Ic-B are respectively seeded into IL LB/Amp liquid medium (composed of 1% peptone (BD company), 0.5% yeast extract (BD company) and 1% NaCl (Sinopharm Chemical Reagent Co,. Ltd.)), and incubated overnight with 180 rpm shaking speed at 37° C.). On the second day, plasmids arc extracted by using an EndoFree Plasmid Maxi Kit (Qiagen, 12381) for transacting HEK293-T cells.

### Example 3: Expressions of the Bifunctional Fusion Protein A and the Bifunctional Fusion protein B

HEK293-T cells in a good growth status with vitality (ratio of viable cells) above 95% are seeded in a 10-layer cell factory (NUNC Company, product number: 140400) in an amount of 1.8×10⁷ cells, and are cultured in a DMEM medium (Coming Company) supplemented with 10% fetal bovine serum (Gibco (Company). The cell factory is mixed thoroughly by inverting and then is placed in a 37° C. incubator at 5% CO₂, and grown for 48 hours. When cells completely adhere to the wall and the confluence reaches 80%. they can be used for a transient transfection.

The plasmids X0GC-CTLA4-anti-IL-17A mAb hc-A and X0GC-anti-IL-17A mAb Ic-A are simultaneously used to transfect cells to express Ore bifunctional fusion protein A. The plasmids X0CC-CTLA4-anti-IL-17A mAb hc-B and X0GC-anti-IL-17A mAb Ic-B are simultaneously used to transfect cells to express the bifunctional fusion protein B. After filtrated with a 0.22 μm filter membrane, 1330 μg each transfection mixture is pipetted and then added to 66 ml serum-free DMEM medium. 2660 μg transaction reagent PEI (Sigma Company) is pipetted and then added to 66 ml serum-free DMEM medium. After mixed respectively, the resulting PET mixture liquid is poured into the DMEM comprising plasmids and then mixed. After standing at room temperature for 15 minutes, the mixture comprising plasmids and PEI is added to 1.3L serum-free DMEM medium, mixed thoroughly and slowly added to the cell factory. The cell factory is placed in a 37° C. incubator at 5% CO₂ and cultured. Four hours later, 266 ml Cell Boost 5 (Thermo Fisher Company) is added and mixed, and then further cultured for 6 days. Centrifuged at 7000 rpm for 20 min, the supernatant is pooled for the following protein purification. The concentrations of the supernatant are determined via ELISA. The protein expression level of each batch of the bifunctional fusion protein A and bifunctional fusion protein B is 4-8 mg/L.

### Example 4: Purification of the Bifunctional Fusion Protein A and the Bifunctional Fusion Protein B

The supernatant from the cell factory is filtered via a 0.2 μm filter membrane. Through ultrafiltration and concentration with a membrane cassette (nominal molecular weight cut off 10 kDa). the solution is replaced with 20 mM a phosphate solution (pH 7.4) comprising 1.50 mM NaCl. Before subjected to a chromatographic column, the supernatant is filtered with a 0.2 μm filter membrane to remove the precipitates. This step is carried out at 4° C.

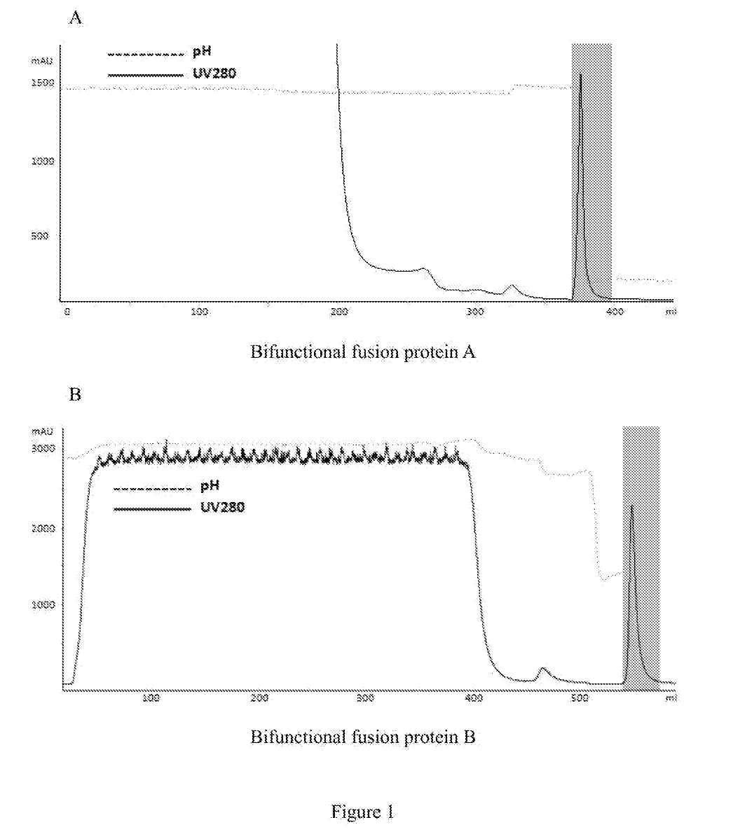

Purification step is carried out at 4° C. by using AKTA explorer 100 type protein purification system (GE Healthcare) and affinity chromatographic column rProtein A Sepharose Fast Flow (16 mm I.D., 22 ml. GE Healthcare). First of all, the chromatographic column is equilibrated with mobile phase A (20 mM sodium phosphate buffer. 150 mM NaCl, pH 7.4), After the baseline becomes stable, the cell supernatant as treated above is loaded onto the column with a flow rate of 5 ml/min, followed by washing with mobile phase A. After that, the column is washed with 5-column volume of mobile phase B1 firstly (the mobile phase A comprising 0.5M arginine). Then, 5-column volume of mobile phase B2 (100 mM citric acid, pH 3.0) is used to elute the column, and elution peaks are collected as protein peaks of interest. The flow rate of the above eluting step is 5 ml/min. Chromatograms are shown in FIGS. 1A and B. The elution peaks indicated are collected (gray areas of FIGS. 1A and B), and pH is adjusted to 5.0 through the addition of 1 M sodium acetate solution.

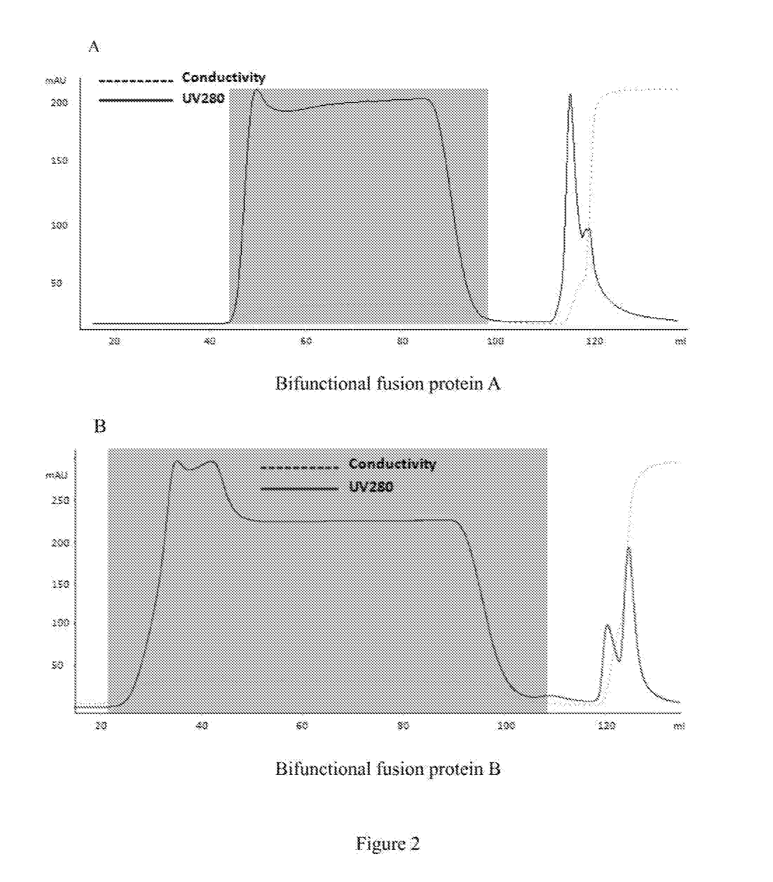

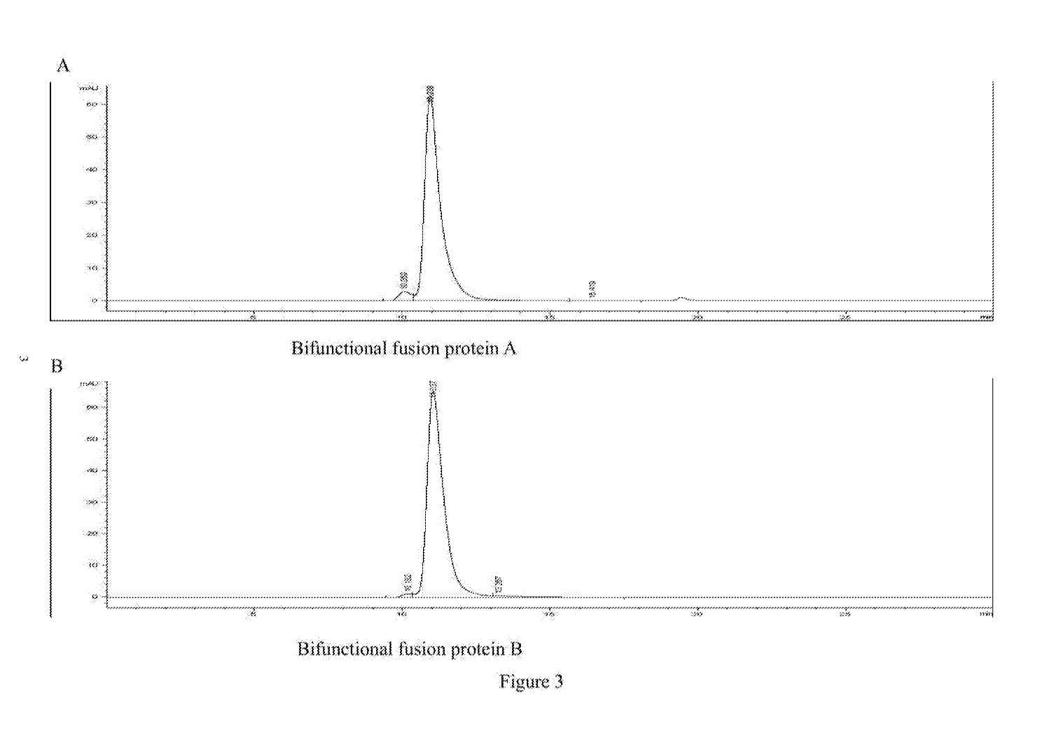

Purification step is carried out at 5° C. by using AKTA explorer 100 type protein purification system (GE Healthcare) and strong anion exchange chromatographic column HiTrap Q Sepharose FF (4 ml, GE Healthcare). First of all the chromatographic column is equilibrated with mobile phase A (20 mM sodium acetate, pH_{5.0}). Alter the baseline becomes stable, the eluent collected and pH adjusted in the above experiment is loaded onto the column with a flow rate of 5 ml/min. Flow-through peaks are collected as protein peaks of interest. After the sample loading, isocratic elution is carried out with mobile phase B (20 mM sodium acetate. l M NaCl, pH 5.0) with a flow rate of 5 ml/min. Chromatograms are shown in FIGS. 2A and B. The fractions indicated are collected (gray areas of FIGS. 2A and B), followed by concentration with a ultrafiltration tube (nominal molecular weight cut off 30 kPa) and aseptic filtration with a 0.2 μm filter membrane in a laminar flow bench. Then an ultraviolet spectrophotometer (280 nm) is used to determine the contents of the protein, followed by purity analysts with a size exclusion high performance liquid chromatography (SE-HPLC). The results are indicated in FIGS. 3A and B. The purities of the bifunctional fusion protein A and the bifunctional fusion protein B are 96.6% and 97.4%, respectively.

### Example 5: Construction of Cell fines Stably Expressing the Bifunctional Fusion Proteins

After thawing, CMO/DG44 (dhfr-) cells are cultured for 2-3 passages. When the survival rate reaches 95%, they are used for the transaction experiments. Transfection of CHO/DG44 cells is carried out with transfection reagent Lipofectamine 2000 (Invitrogen, 11668019) according to the instruction thereof. Specifically, 5 μg of the plasmid and 3 mL α-MEM medium (SAFC, 51451C) are mixed in a centrifuge tube while 20 U L the transfection agent and 3 mL α-MEM medium are mixed in another centrifuge tube. After standing at the laminar flow bench at room temperature for 5 min, the above diluted plasmid and the transfection agent are mixed together and further stand for 30 min. Then 6 mL the mixture is slowly added to a 75 cm² Rectangular Canted Neck cell culture flask (comprising CHO/DG44 cells with a continence of 70-80%) which is then placed in a cell incubator (37° C. and 5% CO₂). After cell growth overnight, the transfection agent mixture is removed, and a fresh α-MEM medium is added followed by adding 10% dialyzed FBS (Gibco, 30067-334). Then, the flask is placed in a cell incubator 37° C. and 5% CO₂) and incubated for 1-2 days. After that, 1 mg/mL G418 (Cellgro. 61-234 RG) is added to the fresh medium, which is used to culture the cells for 2 weeks.

In order to obtain a monoclonal cell strain, a limiting dilution analysis is required. Specifically, a α-MEM medium supplemented with 10% dialyzed FBS and 1 mg/mL G418 is used to dilute the cell suspension to 5 cells/mL which is then seeded to a 96 well plate. The cells are placed in a cell incubator (37° C. and 5% CO₂) for growing till a single cell grows into a single colony. The colonies from single cells are trypsinized and passaged to a 24 well plate. Then the cells are successively passaged to a 12 well plate and a 6 well plate. When the confluence of the monoclonal cell strain in the 6 well plate reaches 90%. the expression level of the protein of interest is measured with ELISA, and clones with a higher expression level are obtained, namely clone No. 52 (14 mg/L), clone No. 85 (56 mg/L) and clone No. 103 (59 mg/L).

### Example 6: Small-Scale Fermentation of the Bifunctional Fusion Proteins

Clone No. 103 is selected for a fermentation in a 5L bioreactor (Sartorius, Bplus twin 5L). It is seeded by 5×10⁵ cells to Hyclone SEM4CHO medium (4 g/L glucose and 0.3 g/L glutamine). The bioreactor has a working volume of 2.4L, and the starting process parameters are as follows: 37° C. pH 7.1. dissolved oxygen 40%-60% and the stirring speed 40 rpm. Afterwards, the stirring speed is increased by 5 rpm per day. Furthermore, after 5 days, the temperature is lowered with a speed of 0.5° C./h until 34° C., and 0.3 mM sodium butyrate is added. Two weeks later, the fermentation broth is harvested and centrifuged at 7000 rpm for 20 min. The supernatant is harvested and kept. Upon ELISA assay, the concentration is determined to be 240 mg/L.

### Example 7: Binding Affinities of the Bifunctional Fusion Proteins A and C to Human IL-17A and Mouse IL-17A

In the binding affinity experiments, the bifunctional fusion protein C is constructed which is similar to the bifunctional fusion proteins A. The amino acid sequence of the bifunctional fusion protein C is set forth in SEQ ID NO. 19 (CTLA4 ECD-linker-anti-IL-17AmAb lc) and SEQ ID NO. 4, that is, CTLA4 ECD is linked to the light chain of anti-IL-17AmAb via a linker (i.e. linked to SEQ ID NO. 5), different from the bifunctional fusion protein A of which CTLA4 ECD is linked to the heavy chain of anti-IL-17AmAb via a linker (i.e. linked to SEQ ID NO. 4). In addition, control antibody A serves as a control antibody of the bifunctional fusion proteins A and C, of which the amino acid sequences are set forth in SEQ ID NO. 4 (the heavy chain of the control antibody A) and SEQ ID NO. 5 (the light chain of the control antibody A). That is, the control antibody A corresponds to the anti-IL-17A antibody moiety in the bifunctional fusion proteins A and C.

The method is specifically implemented as follows. Human IL-17A (Sino Biological Inc., product No. 12047-HNAE) or mouse IL-17A (Sino Biological Inc., product No. 51065-MANE) is coated onto a 96 well high-adsorption ELISA plate (Corning, 2592) with a sodium carbonate solution (15 mM -sodium carbonate, 35 mM sodium bicarbonate, and pH 9.6) with a coating concentration 1 μg/mL, 100 μL/well and coated overnight at 4° C. The plate is washed with PBST (Sigma, product No. P-3563) for 5 times. The plated is blocked with PBST comprising 1% BSA, 300 μL/well, and incubated at 25° C. for at least 1 hour. The plate is washed with PBST for 5 times. Samples to be tested is added at a specific concentration, which are diluted in PBST comprising 1% BSA, 100 μL/well, and incubated at 25° C. for 1 hour. The plate is washed with PBST for 5 times. Then a horse radish peroxidase labeled anti-mouse IgG antibody (1:10,000, in PEST comprising 1% BSA) (Abeam, product No. Ab7068) is added, 100 U L/well, and incubated at 25° C. for 1 hour. The plate is washed with PBST for 5 times. Colorimetric substrate TMB (BD OptEIA, product No. 555214) is added, 100 μL/well, and colorimetric reaction is developed at room temperature for 10 min. 1M H₂SO₄is added with 100 μL/well to terminate the colorimetric development. The Absorbance at 450 nm is read on ELISA.

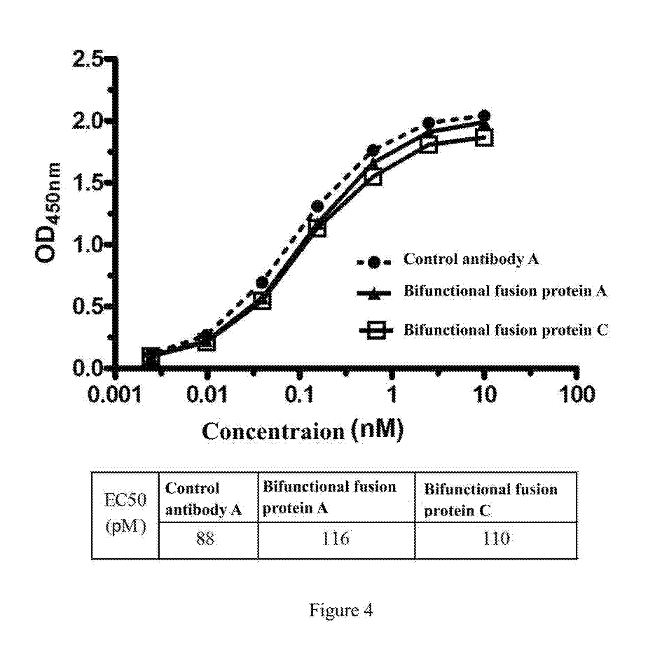

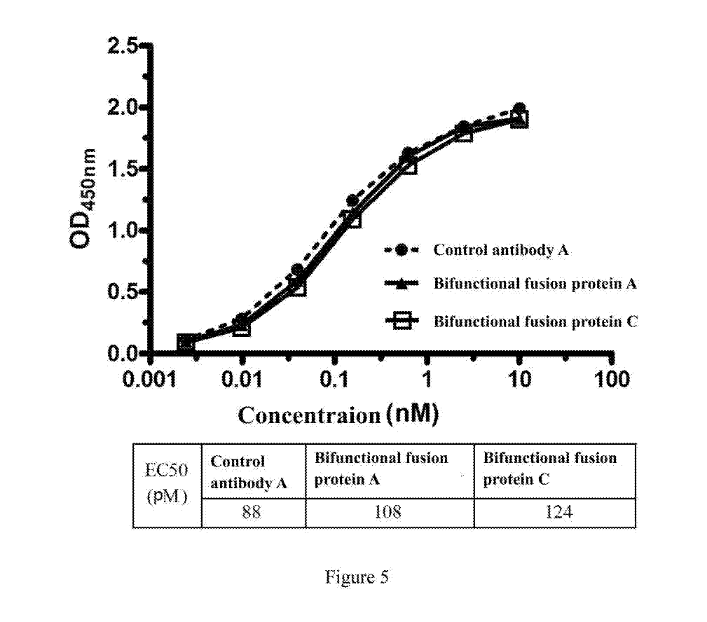

The results are shown in FIGS. 4 and 5. The bifunctional fusion proteins A and C in the present invention have high binding affinities to their antigens human IL-17A (FIG. 4) and mouse IL-17A (FIG. 5), which are similar to that of the control antibody A. The above results show that CTLA4 BCD linked to N-terminal of the heavy chain or light chain of the control antibody A via a tinker does not significantly affect the affinity of the control antibody A to its antigen.

In addition, it shall be noted that the bifunctional fusion protein B in the present invention cannot bind to mouse IL-17A, but has a high binding affinity to human IL-17A (the value measured with ELISA assay in the present invention is EC50=103 pM), which is close to that in U.S. Pat. No. 7,846,443.

### Example 8: Influences of Different Linking Modes of CTLA4 on the Affinity of a Bifunctional Fusion Protein to B7

In order to study and compare the influences of different linking modes of CTLA4 on the affinity of a bifunctional fusion protein to B7, we construct analogues of the bifunctional fusion protein having different CTLA4 linking modes:

(1) The hi functional fusion protein C: as described above, its sequences are SEQ ID NO. 19 (CTLA4 ECD-linker-anti-IL-17 AmAb lc) and SEQ ID NO. 4, i.e.. CTLA4 BCD is linked, to N-terminal of the tight chain of anti-IL 17AmAh via a linker (i.e. linked to SEQ ID NO. 5), different from the bifunctional fusion protein A of which CTLA4 ECD is linked to N-terminal of the heavy chain of anti-IL-17AmAb via a linker (i.e. linked to SEQ ID NO. 4).

(2) The bifunctional fusion protein D: the signal peptide sequence used is set forth in SEQ ID NO.1; the extracellular domain sequence (ECD) of the human CTLA4 protein is set forth in SEQ ID NO. 2; the linker is (G₄S)₅ which has a sequence as set forth in SEQ ID NO. 3; the heavy chain (hc) sequence of the anti-IL-17A antibody is set forth in SEQ ID NO.4 and the light chain (lc) sequence is set forth in SEQ ID NO.5; the amino acid sequence of anti-IL-17AmAb hc-A-linker-CTLA4 ECD is set forth in SEQ ID NO.20. i.e. the CTLA4 of the bifunctional fusion protein D is linked to C-terminal of the heavy chain of the control antibody A via a linker.

(3) The bifunctional fusion protein E: the signal peptide sequence used is set forth in SEQ ID NO.1; the extracellular domain sequence (ECD) of the human CTLA4 protein is set forth in SEQ ID NO.2; the linker is (G₄S)₅ which has a sequence as set forth in SEQ ID NO.3; the heavy chain (hc) sequence of the anti-IL-17A antibody is set forth in SEQ ID NO.4 and the light chain (lc) sequence is set forth in SEQ ID NO.5; the amino acid sequence of anti-IL-17AmAb lc-A-linker-CTLA4 ECD is set forth in SEQ ID NO.21, i.e. the CTLA4 of the bifunctional fusion protein E is linked to C-terminal of the light chain of rive control antibody A via a linker.

CTLA4 ECD can bind to its ligand—B7 molecule. In the experiments for measuring the binding affinity to B7, the binding abilities of the bifunctional fusion proteins A, C, D and E to the B7 molecule are measured on Raji cells which highly express the B7 molecule with flow cytometry This experiment is specifically carried out as follows, Centrifuging at 1000 rpm for 5 min to collect Raji cells (ATCC, product No. CCL-86), washing with PBS (PromoCell, product No. C-40232) comprising 2% FBS (GIBCO, product No. 10099141) once. Resuspending Raji cells by 1×10° C. cells per tube in 200 μL cold PBS which comprises 2% PBS and the samples to be tested at a final concentration of 0.5 nM in a tubes. Incubating the a tubes on ice for 30 min and then washing the cells with PBS comprising 2% FBS twice. Then resuspending the cells in 200 μL cold PBS comprising 2% FBS and an FITC labeled anti-mouse IgG antibody (1:50 diluted). Incubating the tubes on ice in the dark for 30 min and then washing the cells with PBS comprising 2% FBS twice. Resuspending the cells in 500 μL cold PBS comprising 2% FBS and subjecting the cell suspension onto a Flow Cytometer for detection and analysis.

The results are shown in Table 1. The bifunctional fusion protein A has a binding ability to Raji cells highly expressing its ligand—B7 molecule, which is significantly greater than those of the bifunctional fusion proteins C, D and E. The results above show that different linking modes of CTLA4 ECD to the anti-IL-7A antibody may significantly affect its binding ability to B7.

TABLE 1
Binding abilities of the bifunctional fusion proteins to
Raji cells highly expressing B7 molecule

| Samples | Mean fluorescence intensity (MFI) |
|---|---|
| IgG negative control | 5 |
| Bifunctional fusion protein A | 51 |
| Bifunctional fusion protein C | 28 |
| Bifunctional fusion protein D | 9 |
| Bifunctional fusion protein E | 3 |

### Example 9: The Bifunctional Fusion Protein A Inhibits GROα Secretion from HT-29 Cells Induced by IL-17A

Human colorectal cancer cell strain H-29 is from ATCC, HT-29 cells are cultured in RPMI-1640 medium (Gibco, product No. 22400) supplemented with 10% fetal bovine serum (Gibco, product NO. 10099), in a 37° C. cell incubator at 5% CO₂. The cells are passaged twice per week.

The specific procedures for inhibiting GROα secretion induced by IL-17A are as follows. Gradiently diluting the samples to be tested in RPMI 1640 complete medium supplemented with 10% fetal bovine serum, 50 μL per well of the cell culture plate (Coring, product No. 3599). Adding human IL-17A diluted in the same complete medium at a concentration of 240 ng/mL to the cell culture plate, 50 μL per well. Incubating tire cell culture plate in a 37° C. incubator at 5% CO₂. Then resuspending the HT-29 cells in the complete medium and seeding them by 100 μL per well into a 96 well cell culture plate, i.e. about 20,000 cells. Incubating the cells in the 37° C. incubator at 5% CO₂ for 48 h. Centrifuging the cell culture plate at 1000 rpm for 5 min and collecting the culture supernatant. Determining GROα level with a GROα ELISA kit (R&D, product No. DY275) in accordance with tire instruction.

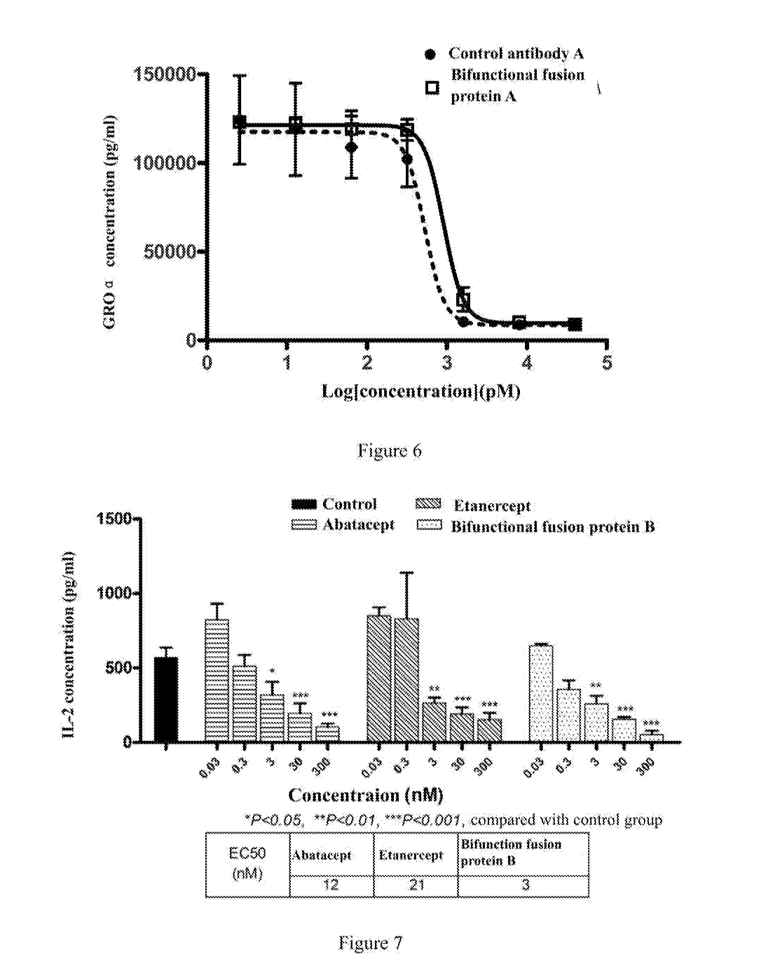

The results are shown in FIG. 6. Both the control antibody A and the bifunctional fusion protein A can inhibit GROα secretion, from HT-29 cells induced by IL-17A, of which IC₅₀ are 532 pM and 911 pM, respectively.

### Example 10: The bifunctional Fusion Protein B Inhibits IL-2 Secretion from Human Peripheral Blood Mononuclear Cells

The immunosuppressive activity of the bifunctional fusion protein B of the present invention, i.e., an activity for inhibiting T lymphocyte activation, is measured with the mixed lymphocyte reaction assay. In brief, two human peripheral blood mononuclear cells (PBMC) samples are obtained from two different individuals, wherein one is inactivated to serve as stimulator cells and the other serves as responder cells. These two PBMC samples are mixed. The stimulator cells can activate immune responses of the responder cells, and secrete a large number of cytokine IL-2. The samples to be tested are co-incubated with this mixed PBMC system and the influence thereof on the IL-2 secretion is determined.

The specific protocol of the mixed lymphocyte reaction assay is as follows. Thawing two PBMC samples from different batches and from different individuals (Lonza, product No. CC-2702) and resuspending them in RPMI-1640 complete medium (Gibco, product No. 22400) supplemented with 10% fetal bovine serum (Gibco, product No. 10099). One PBMC sample serves as stimulator cells, and the other PBMC sample serves as responder cells. Co-incubating the stimulator cells with 50 μg/ml mitomycin C (Wako, product No. 50-07-7) at 37° C. for 45 min and then washing the cells with DEBS (PromoCell, product No. C-40232) twice and then resuspending the cells in RPMI1640 complete medium. Counting the stimulator cells and the responder cells (Innovatis Cedex) and determining cell viability (Trypan blue dye, Invitrogen Company, product No. 15250-061). Then seeding the respondor cells by 50 μL per well (triplicated) to a 96 well cell culture plate (Corning, product No. 3799), i.e. 1×10⁵ cells/well, followed by adding 100 μL the bifunctional fusion protein B at a given concentration, commercialized positive control medicament Abatacept (Orencia™, Bristol-Myers Squibb Company) or Etanercept (Yi Sai Pu™, Shanghai CP Guojian Pharmaceutical Co., Ltd.). Adding 50 μL the stimulator cells to each well by 1×10⁵ cells per well. Incubating the cell culture plate in a 37° C. incubator at 5% CO₂. After 72 hours, pipetting 100 μL culture supernatant from each well and stored at −70° C. Determining IL-2 level with an IL-2 ELISA kit (Raybiotech, product No. ELH-IL2-001) in accordance with the instruction.

The results are shown in FIG. 7. In the mixed human PBMC reaction assay. EC50 of she bifunctional fusion protein B, Abatacept and Etanercept for inhibiting IL-2 secretion are 3 nM, 12 nM and 21 nM. respectively, showing that the bifunctional fusion protein B displays a stronger activity for inhibiting IL-2 secretion than those of the commercialized positive control medicaments Abatacept and Etanercept. Additional tests also show that the bifunctional fusion protein A also displays a stronger activity for inhibiting IL-2 secretion, which is close to tire activity of Abatacept.

### Example 11: Chimeras and Humanized Analogues of the Bifunctional Fusion Protein A

The amino acid sequence of the constant region of the bifunctional fusion protein A, i.e. the amino acid sequence of the constant region of the control antibody A is replaced with a sequence of the constant region of human IgGl The sequence of the heavy chain constant region of human IgGl is set forth in SEQ ID NO. 22, and the sequence of the light chain constant region, is set forth, in SEQ ID NO. 23. A chimera analogue of the bifunctional fusion protein A is obtained therefrom, of which the amino acid sequences are set forth in SEQ ID NO. 24 and SEQ ID NO. 25. The analogue has the reduced mouse-derived ingredients (i.e. sequence) and thus the reduced immunogenicity of the protein.

In addition, partial sequences of SEQ ID NO. 24 and SEQ ID NO. 25 are further modified to replace and mutate the amino acids in the framework region in the variable region thereof so as to further reduce the mouse-derived ingredients. The amino acid sequences of this humanized analogues of the bifunctional fusion protein A obtained therefrom are set forth in SEQ ID NO. 26 and SEQ ID NO. 27.

The antigen binding affinities of the bifunctional fusion protein A, the chimera and the humanized analogues thereof are determined. The results are shown in Table 2. showing that both the chimera and the humanized analogues can effectively retain the antigen binding activity of the parent sequences.

TABLE 2
Binding affinities of the bifunctional fusion protein A, the chimera and
the humanized analogue to human IL-17A and B7

| | | Mean fluorescence |
|---|---|---|
| | EC50 for binding | intensity (MFI) |
| Protein | to IL-17A | for binding to B7 |
| Bifunctional fusion protein A | 201 | 21 |
| Chimera Analogue | 382 | 20 |
| Humanized Analogue | 460 | 20 |

### Example 12: Pharmacodynamics Studies of the Humanized Analogue of the Bifunctional Fusion Protein A in the II Type Collagen Induced Rat Arthritis (CIA) Model

The rat CIA model is a model generally used in the pathogenesis studies of rheumatoid arthritis and screening of therapeutic medicaments. 7 week-old female Wistar rats (purchased from Beijing HFK Bioscience Co., Ltd., P.R. China) are used as the experimental animals. After acclimatization for one week, 5 rats are randomly selected as normal control rats, and the rest of rats are used to establish a rat CIA model. The CIA model is obtained through the primary immunization and booster immunization. As to the primary immunization, 200 μg bovine collagen type II (Chondrex. product No. 20022) and Freund's incomplete adjuvant (Sigma-Aldrich, product No. F5506) are mixed to form an emulsion which is then injected intradermally at the end of the tail of rats. After one week, booster immunization is carried out. With respect to the booster immunization, 100 μg bovine collagen type II and the Freund's incomplete adjuvant are mixed to form an emulsion which is then injected intradermally at the end of the tail of rats. After the booster immunization, when four limbs of the rats are observed to manifest clinical symptoms of arthritis such as redness and edema of the paws, they are randomly divided into groups with 6 rats in each group.

Each group of the CIA model rats are administered with following agents respectively: a solvent (PBS), Abatacept (272 nmol/kg, administered on 1, 5, 8 and 11 day, respectively), a humanized analogue of the bifunctional fusion protein A (33 nmol/kg, administered every other day intraperitoneally, 7 times in total). After administration, pathological changes of the four paws are observed, and the severity of arthritis is scored: 0=no evidence of redness and edema; 1=erythema and mild edema confined to ankle joint or tarsal joint; 2=erythema and mild edema from the ankle joint to the tarsal joint; 3=erythema and moderate edema from the ankle joint to the metatarsal joint; and 4=erythema and severe edema, from the ankle to paws including phalangeal joint or ankylosis of four limbs. Inflammation scores are given to four limbs of the rats, and the maximum score for each rat is 16 scores.

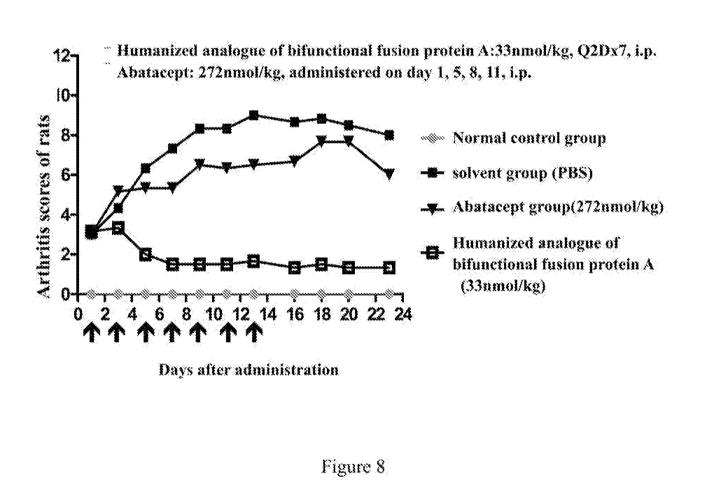

The experimental results are shown in FIG. 8. The rats in the normal control group do not produce inflammatory responses. The CIA model rats in the solvent group produce obvious inflammatory responses. The arthritis scores of the CIA model rats in the group administered with the humanized analogue of the bifunctional fusion protein. A are significantly inhibited, showing a better amelioration effect of inflammation than that in the group administered with Abatacept.

1. A bifunctional fusion protein, comprising the extracellular domain of CTLA4 and an anti-IL-17 antibody.

2-10. (canceled)

11. The bifunctional fusion protein of claim 1, wherein IL-17 is IL-17A.

12. The bifunctional fusion protein of claim 1, further comprising a linker, wherein the extracellular domain of a CTLA4 molecule is linked to N-terminal or C-terminal of the functional fragment which neutralizes the activity of IL-17 via a linker.

13. The bifunctional fusion protein of claim 12, wherein the linker is 1-25 amino acids in length.

14. The bifunctional fusion protein of claim 12, wherein the linker is an amino acid sequence as set forth in SEQ ID NO: 3.

15. The bifunctional fusion protein of claim 1, wherein the functional fragment which neutralizes the activity of IL-17 is an anti-IL-17 antibody or a functional fragment thereof, a chimeric antibody, a humanized antibody, a full humanized antibody, a single-chain antibody or a bispecific antibody.

16. The bifunctional fusion protein of claim 15, wherein the extracellular domain of a CTLA4 molecule is linked to N-terminal of a heavy chain or a light chain of an anti-IL-17 antibody via a linker.

17. The bifunctional fusion protein of claim 15, wherein the anti-IL-17 antibody is an antibody selected from the group consisting of IgG, IgA, IgD, IgE, IgM antibody, and a hybrid thereof.

18. The bifunctional fusion protein of claim 15, wherein the anti-IL-17 antibody is an IgG antibody.

19. The bifunctional fusion protein of claim 1, wherein the extracellular domain of the CTLA4 molecule comprises an amino acid sequence as set forth in SEQ ID NO: 2 or a functional fragment thereof, or an amino acid sequence having the same function derived therefrom through substitution, deletion or addition of one or more amino acid residues, or an amino acid sequence having at least 70% identity thereto and having the same function.

20. The bifunctional fusion protein of claim 15, wherein the heavy chain sequence of the anti-IL-17 antibody comprises an amino acid sequence as set forth in SEQ ID NO: 28, or an amino acid sequence having the same function derived therefrom through substitution, deletion or addition of one or more amino acid residues, or an amino acid sequence having at least 70% identity thereto and having the same function, and wherein the light chain sequence of the anti-IL-17 antibody comprises an amino acid sequence as set forth in SEQ ID NO: 27, or an amino acid sequence having the same function derived therefrom through substitution, deletion or addition of one or more amino acid residues, or an amino acid sequence having at least 70% identity thereto and having the same function.

21. The bifunctional fusion protein of claim 15, wherein the extracellular domain of the CTLA4 molecule as set forth in SEQ ID NO: 2 is linked to N-terminal of the heavy chain sequence of an anti-IL-17 antibody as set forth in SEQ ID NO: 4 or 12 or to N-terminal of the light chain sequence of an anti-IL-17 antibody as set forth in SEQ ID NO: 5 or 13 via a linker as set forth in SEQ ID NO: 3.

22. The bifunctional fusion protein of claim 15, wherein the extracellular domain of the CTLA4 molecule as set forth in SEQ ID NO: 2 is linked to C-terminal of the heavy chain sequence of an anti-IL-17 antibody as set forth in SEQ ID NO: 4 or 12 or to C-terminal of the light chain sequence of an anti-IL-17 antibody as set forth in SEQ ID NO: 5 or 13 via a linker as set forth in SEQ ID NO: 3.

23. The bifunctional fusion protein of claim 1, wherein the amino acid sequence thereof is selected from the group consisting of the following sequence combinations:
a) sequences as set forth in SEQ ID NOs: 5 and 6, SEQ ID NOs: 13 and 14, SEQ ID NOs: 4 and 19, SEQ ID NOs: 24 and 25 and SEQ ID NOs: 26 and 27;
b) amino acid sequences derived from the sequences as set forth in SEQ ID NOs: 5 and 6, SEQ ID NOs: 13 and 14, SEQ ID NOs: 4 and 19, SEQ ID NOs: 24 and 25 and SEQ ID NOs: 26 and 27 through substitution, deletion or addition of one or more amino acid residues, and having an activity for blocking B7 and IL-17A signal pathways simultaneously; or
c) amino acid sequences having at least 70% identity to the sequences as set forth in SEQ ID NOs: 5 and 6, SEQ ID NOs: 13 and 14, SEQ ID NOs: 4 and 19, SEQ ID NOs: 24 and 25 and SEQ ID NOs: 26 and 27, and having an activity for blocking B7 and IL-17A signal pathways simultaneously.

24. A gene encoding the bifunctional fusion protein according to claim 1.

25. The gene of claim 24, wherein the gene comprises nucleotide sequences as set forth in SEQ ID NOs: 7 and 8, or SEQ ID NOs: 15 and 16.

26. A method for preventing, treating or ameliorating a disease, comprising a step of administering a therapeutically effective amount of the bifunctional fusion protein of claim 1 to a subject in need thereof.

27. The method of claim 26, wherein the disease is selected from a group consisting of rheumatoid arthritis, psoriasis, psoriatic arthritis, ankylosing spondylitis, Crohn's disease, systemic lupus erythematosus, lupus nephritis, multiple sclerosis, autoimmune encephalomyelitis, glomerulonephritis, idiopathic thrombocytopenic purpura, primary Sjogren's syndrome, gout, and organ transplantation.

28. The method of claim 26, wherein the disease is selected from a group consisting of rheumatoid arthritis, psoriasis, psoriatic arthritis, ankylosing spondylitis, and Crohn's disease.
